(19) United States
(12) Patent Application Publication
(10) Pub. No.: US 2023/0024759 A1
(43) Pub. Date: Jan. 26, 2023

(54) DETERMINING AN AMOUNT OF NITRIC OXIDE

(71) Applicant: Bedfont Scientific Limited, Harrietsham (GB)

(72) Inventors: Scott DEAMER-SMITH, Harrietsham (GB); Jason Trevor Christopher SMITH, Harrietsham (GB); Hannah VINCE-DREW, Harrietsham (GB)

(21) Appl. No.: 17/777,575

(22) PCT Filed: Nov. 20, 2020
(86) PCT No.: PCT/GB2020/052964

§ 371 Date: May 17, 2022

(30) Foreign Application Priority Data
Nov. 21, 2019 (GB) 1916998.6

(51) Int. Cl. A61B 5/08; G01N 33/497; G01N 33/00; A61B 5/097; A61B 5/087

(52) U.S. Cl. CPC A61B 5/082; G01N 33/497; G01N 33/0044; G01N 33/0054; G01N 33/0037; A61B 5/097; A61B 5/087; G01N 2033/4975; A61B 2010/0087

(57) ABSTRACT

In examples, there is a method comprising receiving an esophageal gas sample at a nitric oxide sensor, the nitric oxide sensor generating a signal indicative of the amount of nitric oxide in the esophageal gas sample, the nitric oxide sensor outputting the signal, and, based on the signal, determining the amount of nitric oxide in the esophageal gas sample.

### BACKGROUND

Nitric oxide is often found in breath samples exhaled from the lungs of asthmatic subjects. This may be tested for, in accordance with the NICE (National Institute for Health and Care Excellence) guidelines, by measuring an amount of nitric oxide in a gas sample exhaled from the lungs at a constant flow rate of 50 millilitres per second (mLs⁻¹).

### BRIEF DESCRIPTION OF THE DRAWINGS

FIG. 1 shows schematically collecting an esophageal gas sample (EGS) according to examples;

FIG. 2 shows schematically transfer of an EGS to apparatus according to examples;

FIG. 3 shows schematically apparatus for determining an amount of nitric oxide in the EGS, according to examples; and

FIG. 4 shows schematically a flow diagram of example methods.

### DETAILED DESCRIPTION

Examples described herein relate to determining an amount of nitric oxide (NO) in an esophageal gas sample (EGS). NO in a human subject's esophagus can be produced by eosinophils in esophagus tissue, which can indicate eosinophilic esophagitis (EoE). Hence, it has been realised that, based on an amount of NO in an esophageal gas sample, it can be determined whether the human subject has the condition EoE.

It is known to take a biopsy of esophageal cells, using an endoscope, to see if eosinophil cells are present in sufficient quantities to determine that the subject has EoE. Such a procedure is invasive, expensive and painful for the subject. Plus, the procedure relies on the skill of the person performing it; if there is inaccuracy in targeting the region from which the cells are taken, the tissue sample may not exhibit eosinophils.

Examples described herein do not require such a biopsy or use of an endoscope. Instead, an esophageal gas sample can be easily, reliably and inexpensively collected and tested to determine the amount of NO. In some examples, a bag is used to collect the esophageal gas sample from a human subject's oral cavity, to reduce or avoid mixing of the sample with gas from the subject's lungs.

Examples are now described in detail with reference to the Figures.

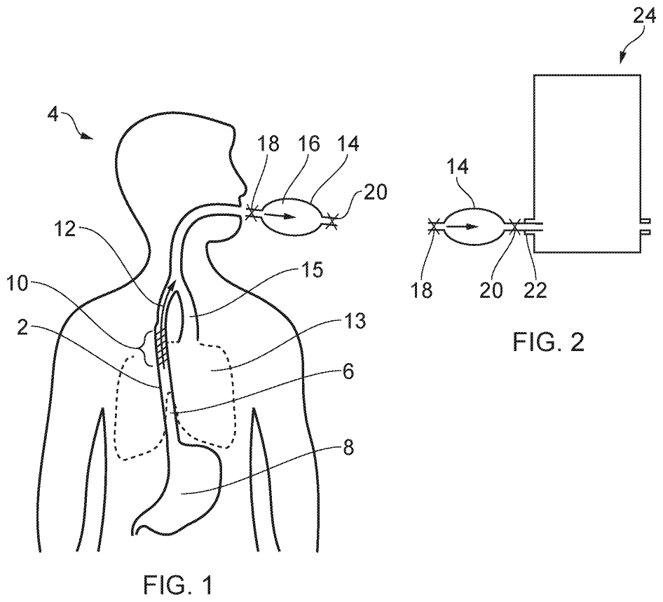

FIG. 1 shows schematically collecting an esophageal gas sample. An esophageal gas sample for example comprises a volume of gas taken from the esophagus 2 of a human subject 4. Although the gas may be taken from the lumen 6 of the esophagus, the gas may have at least partly been emitted by any of the stomach 8, the lower gastrointestinal tract, or eosinophil cells (indicated with shading in FIG. 1) within esophagus tissue. Hence the EGS is for example a mixture of different gases. Gas flow in the Figures is shown by arrows.

Breath samples are typically obtained by collecting gas from the lungs when a human subject exhales. A subject inhales deeply, then exhales for as long as possible. Exhalation is often made into a device with a sensor for determining an amount of a target gas in the breath sample. During such exhalation, the upper esophageal sphincter 12 is closed, so that gas from the esophagus is not collected in such a breath sample. This is useful when determining an amount of a target gas in a lung gas sample, from the lungs 13, via the respiratory tract 15, so that the breath sample is not contaminated with esophageal gas.

In examples described herein, it has been realised how to collect an esophageal gas sample non-invasively. Non-invasively is for example taken to mean that collecting the esophageal gas sample does not require contact with and/or access to the esophagus or upper esophageal sphincter by a device or person assisting with the collection. Examples described herein involve the human subject creating a seal with their lips around an inlet or mouthpiece of for example a container for collecting the gas sample. The inlet or mouthpiece may extend into the subject's oral cavity, at most into the subject's oral cavity proper, via the orifice of the mouth.

It has been further realised in devising examples that by determining an amount of a target gas in an esophageal gas sample, the source of the target gas can be identified. For example, a predetermined minimum amount of nitric oxide in an esophageal gas sample can indicate the presence of eosinophils in esophagus tissue, which in turn can indicate the condition EoE.

In some examples, the esophageal gas sample is collected in a container before then being transferred to apparatus with a nitric oxide sensor for determining an amount of nitric oxide in the gas sample. For example, as shown in FIG. 1, the container 14 comprises a bag 14 or other structure with at least one wall enclosing a void 16 for collecting the esophageal gas sample within. The at least one wall comprises a material substantially impermeable to gas, so the gas sample does not diffuse or leak from the container, and atmospheric gases do not pass into the container, during a reasonable period of time (e.g. 1 hour to 72 hours) before the gas sample is transferred to the nitric oxide sensor.

In some examples the at least one wall is flexible such that the volume of the void is expandable and contractable, whereas in other examples the at least one wall is inflexible, with an inner surface of the at least one wall corresponding to the extent of the void, so the void has a predetermined volume before and after gas collection. The void may be at least partially evacuated (e.g. of gas) before collection of the esophageal gas sample; the pressure difference between the void and the human subject's oral cavity can assist transfer of the esophageal gas sample (EGS) from the oral cavity into the void. Further, with flexible walls, to assist transfer of the EGS from the container into an inlet of apparatus comprising a nitric oxide sensor, the void may be contracted or compressed by squeezing the container, for example its walls.

The container has an inlet 18 for transfer of the EGS into the void. The inlet comprises or is for example a one-way valve for permitting flow of gas through the valve, into the void, but not permitting flow of gas through the valve from the void, so that the EGS may be collected in the container. In some examples the inlet has a cap or cover removable to open the inlet immediately before the EGS is transferred into the void, then replaceable to prevent transfer of extra gas into the void.

The inlet is for example a tube or conduit sealed at one end to the interior of the at least one wall. The other end is for inserting through the orifice of the human subject's mouth, and an outer surface of the tube may be formed to provide a ridge, edge or other structure to act as a stop so the human subject inserts the tube or conduit through the orifice of the mouth, until their lips engage with the ridge, edge or other structure, so that the tube or conduit extends into their oral cavity proper to a desired amount for EGS collection. The ridge, edge or other structure may be shaped to provide a gas tight seal when engaged with the lips. Alternatively, a separate mouthpiece may be fitted to the inlet, to provide the ridge, edge or other structure to act as a stop and/or gas tight seal described above.

In some examples, the container comprises a pump fluidly connected to the inlet. Such a pump may be battery powered and can be activated, when the inlet is inserted in the subject's oral cavity, to initiate or assist transfer of the EGS from the oral cavity of the subject into the void. The pump may be activated and deactivated by a switch, for example by pressing and depressing a button switch, or the duration that the pump is activated for is controlled by a controller (e.g. a processor and connected memory) and is set to be long enough to draw the EGS from the oral cavity, but short enough so as not to draw extra gas from the subject's respiratory tract or lungs which may mix with the EGS.

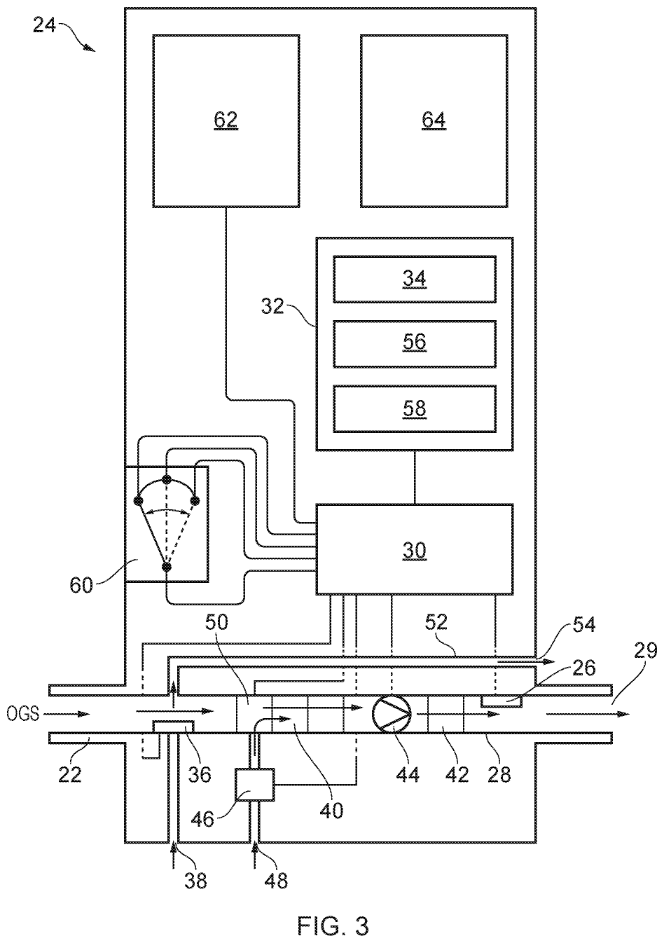

When the EGS is to be transferred to the nitric oxide sensor, this can be done either by the inlet 18 (where the inlet does not comprise a one-way valve, but instead comprises a valve switchable to open or close the lumen for gas to flow in either direction through the conduit or tube) or in other examples via an outlet 20. Such an outlet may, similar to the inlet, comprise a conduit or tube sealed at one end to the interior of the one or more walls. The end of the inlet and/or the end of the outlet distal from the void in examples is shaped to be engageable with an inlet 22 of apparatus 24 (see FIG. 2, with the apparatus described further with FIG. 3) comprising the nitric oxide sensor, so as to fluidly connect the outlet with the inlet, for the EGS then to transfer from the container to the nitric oxide sensor, for example without leakage or loss of the EGS. Transfer of the EGS from the container into the inlet of the apparatus may be assisted by a pump in the apparatus (explained further below using reference numeral 44), which pump may also transfer the EGS to the nitric oxide sensor. The apparatus 24 may be considered to be a device for detecting and measuring an amount of nitric oxide in an EGS. The separate container described herein may be considered as part of apparatus, such as a system, for collecting an EGS and then determining the amount of nitric oxide therein.

FIG. 3 shows schematically apparatus 24 for determining an amount of nitric oxide in an EGS. Such determining may be considered to involve measuring or testing the EGS for the amount of nitric oxide. In examples, the apparatus comprises the inlet 22, a nitric oxide sensor 26 (e.g. available from Membrapor, Birkenweg 2 8304 Wallisellen, Switzerland), a conduit 28 between the inlet and the nitric oxide sensor, at least one processor 30, and at least one memory 32, in other words storage, comprising instructions 34 which, when executed using the at least one processor, cause the apparatus at least to determine, based on a signal output by the nitric oxide sensor, an amount of nitric oxide in an EGS at the nitric oxide sensor. The sensor 26 is calibrated for use as the skilled person will understand, and associated calibration data may be stored in the at least one memory 32. The amount of nitric oxide determined is for example a concentration of nitric oxide in a unit volume of gas, such as 1 millilitre (mL).

Further features of the apparatus are now described for completeness of describing FIG. 3, but it is to be appreciated that in further examples some such features may be implemented differently or absent and/or additional features may be present. Various features are connected by signal lines or electrical connections to the processor 30, as shown by single lines in the Figure; where such lines cross another feature, for example the conduit 28, a portion of the line is dashed.

An EGS enters the apparatus via the inlet 22, into the conduit 28 for conveying the EGS from the inlet to the nitric oxide sensor 26. In some examples, a pressure sensor 36 in the conduit is connected via a port 38 to atmospheric pressure and is for measuring a pressure of an incoming EGS. This pressure measurement is indicative of a flow rate of the EGS, which as explained later influences when to determine an amount of nitric oxide in the EGS. In other examples, such a pressure sensor may be unnecessary, for example if the EGS is transferred from a container such as that described earlier. Such a pressure sensor is an example of a flow rate sensor for determining a flow rate of the EGS exhaled into the inlet.

Along the conduit there may be one or more EGS conditioning elements, to prepare the EGS for the nitric oxide sensor. For example, there is a particulate filter 40 (e.g. to trap particles of 25 micrometres or larger) and a drying element 42 (e.g. a 500 millimetre length of Nafion tube) to reduce or remove water vapour from the EGS. Further, so that the nitric oxide sensor is exposed to zero, or background levels of nitric oxide before being exposed to the EGS, a scrubber 46 (connected via port 48 to the atmosphere) provides gas of the appropriate nitric oxide level to the nitric oxide sensor. Once the EGS has passed the nitric oxide sensor it exits from an outlet 29 of the conduit 28.

A pump 44 in examples moves the EGS from the inlet to the nitric oxide sensor 26, but also moves gas from the scrubber to the nitric oxide sensor 26, depending on a switched position of a two-way (three port) solenoid valve 50. Such a pump can also assist transfer of the EGS from the void of the container to the inlet of the apparatus 22. In some examples, where the EGS is transferred from the human subject's oral cavity into the inlet 22, without first being transferred to the container, the pump 44 or a different pump in the apparatus is configured to initiate or assist transfer of the EGS in a similar manner as explained above for the pump of the container. Thus, the pump can be operated to transfer the EGS into the inlet and possibly further into the conduit, at a suitable flow rate and/or for a suitable duration long enough to transfer the EGS to the inlet but short enough so as not to draw any, or too much, extra gas from the subject's respiratory tract or lungs.

Another conduit 52 branches from the conduit leading to the nitric oxide sensor and instead fluidly connects the inlet to a restrictor port 54, so that a flow rate of the EGS through the conduit 28 to the nitric oxide sensor can be restricted.

In some examples, the apparatus is configured only for a method of determining an amount of nitric oxide in the EGS in a first mode of operation. In further examples, the nitric oxide sensor may also be used in a second or third mode of operation to determine an amount of nitric oxide in a lung gas sample (e.g. to determine if a human subject has asthma) and/or to determine an amount of ammonia in a lung gas sample (e.g. to determine that the amount of ammonia is produced by Helicobacter pylori). Further details will be explained later, and instructions respectively for methods of such second and third modes are labelled 56 and 58. Depending on the determination to be performed, the apparatus may be switched to the first, second and third mode, using a three-way switch 60.

In examples the apparatus comprises a display 62, such as a liquid crystal display panel with associated control electronics, and a power supply such as a battery 64. Electrical connections connect the power supply to the various features using power, as the skilled person will understand, but for clarity those connections are not illustrated.

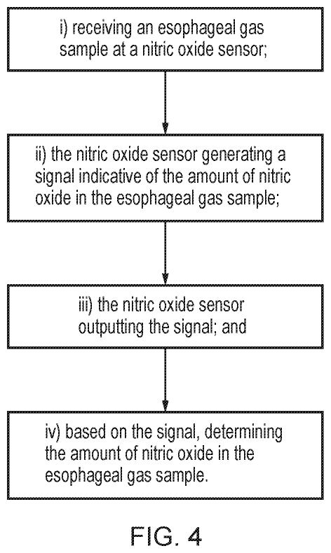

Examples will now be described with reference to FIGS 3 and 4, to explain how an EGS is obtained, collected and then an amount of nitric oxide in the EGS determined.

Note that various parts of the method are implemented by executing appropriate instructions stored in the at least one memory 32; it is to be understood that the at least one memory in examples described herein stores the relevant instructions for such parts of the method, including those referred to later with reference numerals 56 and 58.

In examples, to collect an EGS, gas from the esophagus is transferred from the esophagus to the oral cavity of the human subject. This can be achieved by causing the human subject to eructate (otherwise known as to burp or belch). For example, the human subject swallows a substance in sufficient quantity to initiate output of gas from the stomach. The substance may generate gas upon reaction or dissolution with stomach acid (and is e.g. sodium bicarbonate) or instead may be effervescent, so that the substance comprises gas which it emits (e.g. carbonated water). The gas generated or emitted from the stomach initiates output of the EGS to pass from the esophagus, through the upper esophageal sphincter (which as part of eructation is caused to relax), and into the oral cavity. The EGS may therefore also comprise the gas generated or emitted from the stomach, but as the substance does not emit the target gas (such as nitric oxide) in the EGS, this does not interfere in determining the amount of target gas in the EGS. Before eructation, the subject may already have an inlet or mouthpiece either of a container such as that described above, or of the apparatus comprising the nitric oxide sensor, in their oral cavity for collecting the EGS. Thus, upon and during eructation, the EGS is transferred from the oral cavity into the inlet immediately by the pressure of the EGS being expelled during eructation. Or, in other examples, after eructation, the subject holds the EGS in their oral cavity with their lips sealed, inserts the inlet or mouthpiece through their mouth, and then transfers the EGS into the inlet by for example squeezing their cheeks together, to expel the EGS. Either of these approaches to collecting the EGS reduces or avoids mixing of the EGS with gas from the human subject's lungs.

In other examples, where the subject transfers the EGS from the oral cavity directly into the inlet of the apparatus, without collecting it in a container first, the subject eructates in a similar manner as described above then exhales gas from their lungs to assist transfer of the EGS from the oral cavity into the inlet. The exhalation is at a particular flow rate, less than 40 mLs⁻¹, so that the amount of nitric oxide in the EGS can be determined by the nitric oxide sensor for example before enough gas exhaled from the lungs reaches the nitric oxide sensor which might interfere with determining the amount of nitric oxide in the EGS. Otherwise, any nitric oxide in the exhaled gas from the lungs may interfere with determining the amount of nitric oxide in the EGS.

During collecting the EGS, a nose clip may be worn by the subject, to prevent loss of the EGS via the nasal cavity and also to reduce or avoid atmospheric gas mixing with the EGS.

Once the EGS has entered the inlet 22 of the apparatus, it passes along the conduit 28 towards the nitric oxide sensor 26, assisted by the pump 44, and via conditioning elements such as the filter 40 and drying element 42. In examples with the solenoid 40, the solenoid 40 is switched so that the EGS can pass to the nitric oxide sensor instead of gas from the scrubber 46.

In method examples described herein and in accordance with FIG. 4, once the EGS is received at the nitric oxide sensor according to i), ii) the nitric oxide sensor generates a signal indicative of the amount of nitric oxide in the EGS. Then, iii), the nitric oxide sensor outputs the signal, for example to the at least one processor, and, iv) based on the signal, the amount of nitric oxide in the EGS is determined, for example using data stored in the at least one memory 32 which correlates different signal levels with different amounts of nitric oxide. The nitric oxide in the EGS in examples is produced by eosinophils in esophagus tissue.

In examples where the EGS is transferred to the inlet 22 from the container, the apparatus may or may not have the flow rate sensor such as the pressure sensor 36. The EGS may be transferred into the inlet at an appropriate flow rate and for a duration required for the nitric oxide sensor to determine the amount of nitric oxide in the EGS. The appropriate flow rate and duration can be set for example by a cross sectional area of the container outlet and/or of the inlet 22, in other examples by operating the pump 44 at an appropriate speed, and/or in other examples by appropriate configuration of the restrictor port. In other examples, with the flow rate sensor such as the pressure sensor, the container may be squeezed to expel the EGS at the appropriate flow rate and for the appropriate duration of time. Assuming the flow rate and/or duration are suitable for determining the amount of nitric oxide in the EGS, an amount of nitric oxide in the EGS is visually and/or audibly indicated, respectively, for example by the display 62 or a light, or an audio output device of the apparatus. In some examples, it is further determined that the amount of nitric oxide in the EGS is indicative of EoE (for example by comparing the amount of nitric oxide against a threshold value stored in the memory 34 which is a minimum amount of nitric oxide indicative of EoE), and it is visually and/or audibly indicated that the amount of nitric oxide in the EGS is indicative of EoE.

In other examples, where the EGS is transferred into the inlet by the human subject exhaling gas from their lungs, the apparatus has the flow rate sensor such as the pressure sensor 36 to determine a flow rate of gas entering the inlet, dependent on the rate of exhalation from the human subject's lungs. In such examples, the method therefore receives, by such exhalation, the EGS at the inlet of the apparatus comprising the nitric oxide sensor. A value of the flow rate is determined by the flow rate sensor, and it is then determined, for example by comparing the value against a threshold value stored in the memory 34, that the value of the flow rate is suitable for (e.g. less than 40 mLs⁻¹) determining the amount of nitric oxide in accordance with iv) to indicate nitric oxide produced by eosinophils in esophagus tissue. If the flow rate is not less than 40 mLs⁻¹, for example is greater than 45 mLs⁻¹, the EGS may pass too quickly to, and past, the nitric oxide sensor; in such a situation, any amount of nitric oxide determined may instead have come from the lungs of the subject, and therefore cannot be used to determine nitric oxide production by eosinophils in the esophagus. If the value of the flow rate is determined to be suitable, an amount of nitric oxide in the EGS is visually and/or audibly indicated, respectively for example by the display 62 or a light, or the audio output device of the apparatus. In some examples, it is further determined that the amount of nitric oxide in the EGS is indicative of EoE (for example by comparing the amount of nitric oxide against a threshold value stored in the memory 34 which is a minimum amount of nitric oxide indicative of EoE), and it is visually and/or audibly indicated that the amount of nitric oxide in the EGS is indicative of EoE.

On the other hand, if the value of the flow rate is not suitable (e.g. greater than 45 mLs⁻¹), the instructions 34 may cause an amount of nitric oxide detected by the sensor and/or an indication of EoE not to be visually and/or audibly indicated.

In other examples, a value of a different exhalation parameter than the flow rate is used to determine whether the flow rate of incoming gas to the inlet is suitable for determining the amount of nitric oxide in accordance with iv).

In addition to determining that the value of an exhalation parameter such as the flow rate is for determining the amount of nitric oxide in accordance with iv), in some examples a duration that the value of the exhalation parameter is maintained for is determined, then it is determined (for example by comparing the value against a threshold value stored in the memory 34) that the duration is long enough for determining the amount of nitric oxide in accordance with iv) to indicate nitric oxide produced by eosinophils in esophagus tissue. If the duration is not long enough, the signal from the sensor may not be maintained for long enough to accurately determine if nitric oxide is produced by eosinophils in the esophagus. If the duration is long enough, an amount of nitric oxide in the EGS and/or an indication of EoE is visually and/or audibly indicated, respectively for example by the display 62 or a light, or the audio output device of the apparatus.

In examples with the apparatus comprising the exhalation parameter sensor such as the pressure sensor, an audible and/or visual indication of the exhalation parameter such as the current flow rate of the EGS received by the inlet may be given. In this way it is indicated whether the flow rate is suitable or not (e.g. too high or too low) for determining the amount of nitric oxide in the EGS, and a duration that a suitable flow rate has been maintained for. Such audible and/or visual feedback can be used by the human subject to adjust their exhalation rate to ensure the EGS is received by the inlet at the appropriate flow rate and for long enough.

As well as using the nitric oxide sensor to determine an amount of nitric oxide in the EGS, in the first mode in accordance with instructions 34, in some examples the apparatus is also configured, in the second mode in accordance with instructions 56, to determine an amount of nitric oxide in a lung gas sample by v) receiving a lung gas sample at the nitric oxide sensor; vi) the nitric oxide sensor generating a signal indicative of the amount of nitric oxide in the lung gas sample; vii) the nitric oxide sensor outputting the signal of vi); and viii) based on the signal of vi), determining the amount of nitric oxide in the lung gas sample. In a similar way as described earlier that a value of the flow rate is determined as suitable for determining an amount of nitric oxide in the EGS, the value of the exhalation parameter such as the flow rate is determined as suitable for (e.g. at least 45 mLs⁻¹) determining the amount of nitric oxide in accordance with viii) to indicate nitric oxide produced by lung tissue. The amount of nitric oxide in the lung gas sample of v) may be audibly and/or or visually indicated by the apparatus, and in some examples the method comprises determining that the amount of nitric oxide in the lung gas sample of v) is indicative of asthma, and at least one of audibly or visually indicating that the amount of nitric oxide in the gas sample of v) is indicative of asthma.

In other examples, as an alternative second mode, or (in addition to the first and second modes) in a third mode in accordance with instructions 58, the apparatus is also configured to determine an amount of ammonia in a lung gas sample by ix) receiving a lung gas sample at a converter for converting ammonia to nitric oxide; x) converting, by the converter, ammonia in the lung gas sample of ix) to nitric oxide; xi) receiving, at the nitric oxide sensor, nitric oxide converted from ammonia in the lung gas sample of ix); xii) the nitric oxide sensor generating a signal indicative of the amount of nitric oxide converted from ammonia in the lung gas sample of ix); xiii) the nitric oxide sensor outputting the signal of x); and xiv) based on the signal of x), determining the amount of ammonia in the lung gas sample of ix). The converter is for example part of the apparatus and comprises a sufficiently large surface which is heatable by a heating element to a high enough temperature for cracking the ammonia in the lung gas sample to nitric oxide. The amount of ammonia in the lung gas sample of ix) may be audibly and/or or visually indicated by the apparatus, and in some examples the method comprises determining that the amount of ammonia in the lung gas sample of ix) is indicative of the presence of Helicobacter pylori, and at least one of audibly or visually indicating that the amount of ammonia in the gas sample of ix) indicates production by Helicobacter pylori.

Where the apparatus is operable in the first, second and third modes, the apparatus is switchable between modes in some examples using the three-way switch. In other examples, where the apparatus is operable in first and second modes, but not in the third mode, a two-way switch is used instead. Although such switches are described as hardware switches, they may instead be implemented using software and a touch sensitive implementation of the display.

It is to be appreciated that the apparatus 24 may comprise other features for operation (not illustrated), for example display panel control circuitry which the skilled person is familiar with. The at least one processor is for example a general purpose processor, a microprocessor, a digital signal processor (DSP), an application specific integrated circuit (ASIC), a field programmable gate array (FPGA) or other programmable logic device, a discrete gate or transistor logic, discrete hardware components, or any suitable combination thereof designed to perform the functions described herein. A processor may also be implemented as a combination of computing devices, e.g., a combination of a DSP and a microprocessor, a plurality of microprocessors, one or more microprocessors in conjunction with a DSP core, or any other such configuration. The processor may be coupled, via one or more buses, to read information from or write information to the at least one memory. The at least one processor may additionally, or in the alternative, contain memory, such as processor registers.

Further, the apparatus may comprise a communications subsystem, configured for the apparatus to communicate with for example a computing device via a data network, for example a computer network such as the Internet, a local area network, a wide area network, a telecommunications network, a wired network, a wireless network, or some other type of network. The communications subsystem may further or alternatively for example comprise an input/output (I/O) interface, such as a universal serial bus (USB) connection, a Bluetooth or infrared connection, or a data network interface for connecting the apparatus to a data network such as any of those described above.

There may be a user input subsystem, comprising for example an input device for receiving input from a user of the apparatus. Example input devices include, but are not limited to, a keyboard, a rollerball, buttons, keys, switches, a pointing device, a mouse, a joystick, a remote control, an infrared detector, a voice recognition system, a bar code reader, a scanner, a video camera (possibly coupled with video processing software to, e.g., detect hand gestures or facial gestures), a motion detector, a microphone (possibly coupled to audio processing software to, e.g., detect voice commands), or other device capable of transmitting information from a user to the device. The input device may also take the form of a touch-sensitive panel associated with the display, in which case a user responds to prompts on the display by touch. The user may enter textual information through the input device such as the keyboard or the touch-sensitive display.

The apparatus may also include a user output subsystem (not illustrated) including for example an output device for providing output to a user of the apparatus. Examples include, but are not limited to, an audio output device including for example one or more speakers, headphones, earphones, alarms, or haptic output devices. The output device may be a connector port for connecting to one of the other output devices described, such as earphones. The display is also considered an output device for displaying graphics, images and/or text to a user.

The apparatus may comprise a power subsystem with power circuitry for use in transferring and controlling power consumed by the apparatus. The power may be provided by a mains electricity supply or from the battery 64, via the power circuitry. The power circuitry may further be used for charging the battery from a mains electricity supply.

System storage includes the at least one memory 32, for example at least one of volatile memory and non-volatile memory and may comprise a non-transitory computer readable storage medium. The volatile memory is for example a Random Access Memory (RAM). The non-volatile (NV) memory is for example be a solid state drive (SSD) such as Flash memory, or Read Only Memory (ROM). Further storage technologies may be used, for example magnetic, optical or tape media, compact disc (CD), digital versatile disc (DVD), Blu-ray or other data storage media. The volatile and/or non-volatile memory may be removable or non-removable.

Any of the at least one memory stores data for controlling the apparatus, for example components or subsystems of the apparatus. Such data may for example be in the form of computer readable and/or executable instructions, for example computer program instructions. Therefore, the at least one memory and the computer program instructions may be configured to, with the at least one processor, cause the apparatus to perform any of the methods described herein.

For example, the at least one memory stores display data indicative of graphics, images and/or text to be output provided by the display 62. The at least one memory may store program data representing computer executable instructions, for example in the form of computer software, for the apparatus to run applications or program modules for the apparatus or components or subsystems of the apparatus to perform certain functions or tasks, and/or for controlling components or subsystems of the apparatus. For example, application or program module data includes any of routines, programs, objects, components, data structures or similar.

In examples described above, the nitric oxide sensor used for the first, second and third modes is the same. However, it is envisaged in other examples that a different nitric oxide sensor than that for the first mode may be used for the second or third mode.

Examples described above relate to determining an amount of nitric oxide in an EGS. It is further envisaged that in other examples the target gas is not nitric oxide, but is a different target gas in the EGS. Hence, in further examples the method comprises i) receiving an esophageal gas sample at a target gas sensor; ii) the target gas sensor generating a signal indicative of the amount of a target gas in the esophageal gas sample; iii) the target gas sensor outputting the signal; and iv) based on the signal, determining the amount of the target gas in the esophageal gas sample. The target gas is for example at least one of: ammonia, hydrogen sulfide, or methane and, in a similar manner to previous examples described for nitric oxide, an amount of the target gas may be determined, for example to indicate if the subject has a particular condition.

The above examples are to be understood as illustrative. Further examples are envisaged. It is to be understood that any feature described in relation to any one example may be used alone, or in combination with other features described, and may also be used in combination with one or more features of any other of the examples, or any combination of any other of the examples. Furthermore, equivalents and modifications not described above may also be employed without departing from the scope of the accompanying claims.

1. A method comprising:
i) receiving an esophageal gas sample at a nitric oxide sensor;
ii) the nitric oxide sensor generating a signal indicative of the amount of nitric oxide in the esophageal gas sample;
iii) the nitric oxide sensor outputting the signal; and
iv) based on the signal, determining the amount of nitric oxide in the esophageal gas sample.

2. The method of claim 1, wherein at least one of:
nitric oxide in the esophageal gas sample is produced by eosinophils in esophagus tissue; or
the method comprises non-invasively collecting the esophageal gas sample.

3. (canceled)

4. The method of claim 1, comprising:
before i), collecting the esophageal gas sample in a container;
fluidly connecting an outlet of the container with an inlet of apparatus comprising the nitric oxide sensor; and
transferring the esophageal gas sample from the container to the nitric oxide sensor.

5. The method of claim 4, wherein at least one of:
a pump of the apparatus assists transferring the esophageal gas sample from the container to the nitric oxide sensor;
the container comprises a bag with at least one flexible wall substantially impermeable to gas;
the method comprises squeezing the container to assist transferring the esophageal gas sample from the container to the nitric oxide sensor;
the method comprises the esophageal gas sample transferring from a human subject's oral cavity into an at least partially evacuated void of the container; or
the method comprises the esophageal gas sample transferring from the human subject's oral cavity into an at least partially evacuated void of the container via a one-way valve.

6. (canceled)

7. (canceled)

8. (canceled)

9. (canceled)

10. The method of claim 1, comprising collecting the esophageal gas sample after the esophageal gas sample has passed from the esophagus and through the upper esophageal sphincter.

11. The method of claim 10, comprising initiating output of the esophageal gas sample from the esophagus, through the upper esophageal sphincter, by the human subject swallowing a substance to initiate output of gas from the stomach.

12. The method of claim 1, comprising:
receiving, by exhalation from a human subject's lungs, the esophageal gas sample at an inlet of apparatus comprising the nitric oxide sensor;
determining a value of an exhalation parameter of the esophageal gas sample;
determining that the value of the exhalation parameter is for determining the amount of nitric oxide in accordance with iv) to indicate nitric oxide produced by eosinophils in esophagus tissue.

13. The method of claim 12, wherein at least one of:
the value of the exhalation parameter is indicative of a flow rate value of the esophageal gas sample;
the value of the exhalation parameter indicative of a flow rate value is less than 40 mLs⁻¹; or
the method comprises:
determining a duration that the value of the exhalation parameter is maintained for, and
determining that the duration is long enough for determining the amount of nitric oxide in accordance with iv) to indicate nitric oxide produced by eosinophils in esophagus tissue.

14. (canceled)

15. (canceled)

16. The method of claim 1, comprising at least one of:
at least one of audibly or visually indicating the amount of nitric oxide in the esophageal gas sample;
or
determining that the amount of nitric oxide in the esophageal gas sample is indicative of EoE, and at least one of audibly or visually indicating that the amount of nitric oxide in the esophageal gas sample is indicative of EoE.

17. (canceled)

18. The method of claim 1, comprising:
v) receiving a lung gas sample at the nitric oxide sensor;
vi) the nitric oxide sensor generating a signal indicative of the amount of nitric oxide in the lung gas sample;
vii) the nitric oxide sensor outputting the signal of vi); and
viii) based on the signal of vi), determining the amount of nitric oxide in the lung gas sample.

19. The method of claim 18, comprising:
determining a value of an exhalation parameter of the lung gas sample of v);
determining that the value of the exhalation parameter of the lung gas sample of v) is for determining the amount of nitric oxide in accordance with viii) to indicate nitric oxide produced by lung tissue.

20. The method of claim 19, the value of the exhalation parameter of the lung gas sample of v) indicative of a flow rate value of at least 45 mLs⁻¹.

21. The method of claim 18, comprising at least one of:
at least one of audibly or visually indicating the amount of nitric oxide in the lung gas sample of v);
or
determining that the amount of nitric oxide in the lung gas sample of v) is indicative of asthma, and at least one of audibly or visually indicating that the amount of nitric oxide in the gas sample of v) is indicative of asthma.

22. (canceled)

23. The method of claim 1, comprising:
ix) receiving a lung gas sample at a converter for converting ammonia to nitric oxide;
x) converting, by the converter, ammonia in the lung gas sample of ix) to nitric oxide;
xi) receiving, at the nitric oxide sensor, nitric oxide converted from ammonia in the lung gas sample of ix);
xii) the nitric oxide sensor generating a signal indicative of the amount of nitric oxide converted from ammonia in the lung gas sample of ix);
xiii) the nitric oxide sensor outputting the signal of x); and
xiv) based on the signal of x), determining the amount of ammonia in the lung gas sample of ix).

24. The method of claim 23, comprising at least one of:
at least one of audibly or visually indicating the amount of ammonia in the lung gas sample of ix);
or
determining that the amount of ammonia in the lung gas sample of ix) is indicative of production by Helicobacter pylori, and at least one of audibly or visually indicating that the amount of ammonia in the lung gas sample of xi) indicates production by Helicobacter pylori.

25. (canceled)

26. Apparatus comprising:
an inlet;
a nitric oxide sensor;
a conduit between the inlet and the nitric oxide sensor;
at least one processor; and
at least one memory comprising instructions which, when executed using the at least one processor, cause the apparatus to i) determine, based on a signal output by the nitric oxide sensor, an amount of nitric oxide in an esophageal gas sample at the nitric oxide sensor.

27. The apparatus of claim 26, comprising at least one of:
a container comprising an outlet engageable with the inlet for transfer of the esophageal gas sample from the container to the nitric oxide sensor;
a pump configured to assist transfer of the esophageal gas sample from the container or from a human subject's oral cavity to the nitric oxide sensor; or
a flow rate sensor to determine a flow rate of the esophageal gas sample exhaled into the inlet.

28. (canceled)

29. The apparatus of claim 27, wherein at least one of:
a void of the container before collection of the esophageal gas sample in the container is at least partially evacuated;
the container comprises a bag with at least one flexible wall substantially impermeable to gas; or
the container comprises a one-way valve for collection of the esophageal gas sample in the container.

30. (canceled)

31. (canceled)

32. (canceled)

33. The apparatus of claim 26, wherein the instructions, when executed using the at least one processor, cause the apparatus to:
determine using the flow rate sensor that the flow rate of the esophageal gas sample is less than 40 mLs⁻¹;
determine the amount of nitric oxide in the esophageal gas sample.

34. The apparatus of claim 33, wherein the instructions, when executed using the at least one processor, cause the apparatus to:
at least one of:
audibly or visually indicate the amount of nitric oxide in the esophageal gas sample; or
audibly or visually indicate that the amount of nitric oxide in the esophageal gas sample is indicative of EoE.

35. The apparatus of claim 26, switchable between at least two of:
a first mode configured for determining, in accordance with i), the amount of nitric oxide in the esophageal gas sample;
a second mode configured for determining an amount of nitric oxide in a first lung gas sample received at the nitric oxide sensor; or
a third mode configured for determining an amount of nitric oxide converted from ammonia in a second lung gas sample received at the nitric oxide sensor.

36. The apparatus of claim 35,
the first mode configured to at least one of audibly or visually indicate that the amount of nitric oxide in the esophageal gas sample is indicative of EoE;
the second mode configured to at least one of audibly or visually indicate that the amount of nitric oxide in the first lung gas sample is indicative of asthma; and
the third mode configured to at least one of audibly or visually indicate that the amount of ammonia in the second lung gas sample is indicative of Helicobacter pylori.

37. A method comprising:
i) receiving an esophageal gas sample at a target gas sensor;
ii) the target gas sensor generating a signal indicative of the amount of a target gas in the esophageal gas sample;
iii) the target gas sensor outputting the signal; and
iv) based on the signal, determining the amount of the target gas in the esophageal gas sample.

38. The method of claim 37, wherein the target gas is at least one of: ammonia, nitric oxide, hydrogen sulfide, or methane.
